(19) United States
(12) Patent Application Publication
(10) Pub. No.: US 2011/0092454 A1
(43) Pub. Date: Apr. 21, 2011

(54) Methods For Treating Mycoplasma Related Conditions In Livestock

(76) Inventor: Dean Rimoldi, Tonapah, AZ (US)

(21) Appl. No.: 12/895,336

(22) Filed: Sep. 30, 2010

Related U.S. Application Data
(60) Provisional application No. 61/247,129, filed on Sep. 30, 2009.

(51) Int. Cl. A61K 31/70; A61K 31/56; A61P 31/04

(52) U.S. Cl. 514/52; 514/171

(57) ABSTRACT

The present invention provides methods and related kits for treating or preventing a mycoplasma-related bovine disease. In one aspect, for example, a method for treating or preventing a mycoplasma-related bovine disease may include administering to a bovine a therapeutically effective amount of a steroid or a pharmaceutically acceptable salt thereof, and administering to the bovine a therapeutically effective amount of at least one antibiotic.

### RELATED CASES

This application claims priority of U.S. Provisional Patent Application Ser. No. 61/247,129, filed Sept. 30, 2009, which is hereby incorporated herein by reference in its entirety.

### FIELD OF THE INVENTION

The present invention relates to the treatment of mycoplasma related conditions in cattle. Accordingly, this invention involves the fields of biology, veterinarian medicine, and pharmaceutics.

### BACKGROUND OF THE INVENTION

Mycoplasma related diseases are contagious conditions that can result in significant loss in cattle herds due to culling and death. These conditions are a result of infection by a variety of mycoplasma bacteria. A few examples of bacterial species that have been isolated from cattle include Mycoplasma bovis, M. californicum, M. canadense, M. bovigenitalium, M. alkalescens, M. arginini, M. bovihirnis, M. dispar, bovine group 7, and F-38. Bacterial species from Ureaplasma and Acholeplasma have also been isolated, and are often referred to as mycoplasmas. It is generally believed that mycoplasmas play a secondary role in infections, most often exacerbating pre-existing disease. However, it has also been suggested that M. bovis and possibly other mycoplasmas can be a primary cause of disease. M. bovis may be considered one of the more pathogenic species, and is frequently associated with pneumonia, mastitis, and arthritis in cattle. M. bovis may also be associated with meningitis, otitis media, kerato-conjunctivitis, decubital abscesses, vaginitis, and abortion.

As long as mycoplasma remains in the upper respiratory tract it appears to cause no clinical signs of disease. Entry of the bacterium into the lungs, however, causes a pneumonia that is somewhat different from the shipping fever pneumonia that most beef producers recognize. From the respiratory tract a mycoplasma such as M. bovis can travel through the bloodstream, and thus often enters joints, organs, and nerves. In beef cattle, mycoplasma most commonly goes to the joints where it can cause a crippling arthritis and tenosynovitis. Mycoplasmas can also infect the ears, eyes, udders, and genital tract. The treatment of mycoplasma related conditions has proven difficult, in part, because mycoplasma pathogens lack a cell wall, and are thus resistant to many commonly used antibiotics.

### SUMMARY OF THE INVENTION

The present invention provides methods and related kits for treating or preventing a mycoplasma-related bovine disease. In one aspect, for example, a method for treating or preventing a mycoplasma-related bovine disease may include administering to a bovine a therapeutically effective amount of a steroid or a pharmaceutically acceptable salt thereof, and administering to the bovine a therapeutically effective amount of an antibiotic. In one specific aspect, the steroid can be a glucocorticoid steroid. Nonlimiting examples of glucocorticoid steroids can include hydrocortisone, cortisone acetate, prednisone, prednisolone, methylprednisolone, dexamethasone, betamethasone, triamcinolone, beclometasone, fludrocortisone acetate, deoxycorticosterone acetate, aldosterone, and combinations thereof. In one specific aspect, the glucocorticoid steroid is dexamethasone.

A variety of antibiotics are contemplated for use in the treatments according to aspects of the present invention. For example, the antibiotic may include a β-lactam, a quinolone, a tetracycline, a sulfonamide, a fenicole, a macrolide, and the like. In one specific aspect, the antibiotic is a penicillin antibiotic. Additionally, combinations of multiple antibiotics can be coadministered with the steroid. For example, in one aspect a β-lactam antibiotic and a sulfonamide antibiotic can be coadministered with the steroid. In a more specific aspect, the β-lactam is a penicillin antibiotic and the sulfonamide is sulfadimethoxine.

The bovine may also receive a steroid coadministered with at least one of florfenicol, thiamphenicol, chloramphenicol, and combinations thereof. Additionally, in one aspect, a therapeutically effective amount of tulathromycin can be coadministered with the steroid to the bovine. Furthermore, a vitamin compound such as a Vitamin B compound can be coadministered with the steroid.

In a more specific aspect, a method for treating or preventing a mycoplasma-related bovine disease can include administering to a bovine a therapeutically effective amount of dexamethasone or a pharmaceutically acceptable salt thereof, a penicillin antibiotic, sulfadimethoxine, and at least one of florfenicol, thiamphenicol, chloramphenicol, and tulathromycin. Furthermore, in one aspect a therapeutically effective amount of a Vitamin B compound or a Vitamin B complex can additionally be administered.

The present invention additionally contemplates a kit for treating or preventing a mycoplasma-related bovine disease. Such a kit may include a housing containing a steroid, at least one antibiotic, and a set of instructions describing a use of the steroid and the at least one antibiotic to treat or prevent the mycoplasma-related bovine disease. In one specific aspect the steroid is dexamethasone. In another specific aspect, the at least one antibiotic includes a β-lactam. In yet another specific aspect, the at least one antibiotic includes at least one of florfenicol and tulathromycin. In a further aspect, the at least one antibiotic includes sulfadimethoxine. In yet a further aspect, the kit may include a Vitamin B complex.

### DETAILED DESCRIPTION

Before the present invention is disclosed and described, it is to be understood that this invention is not limited to the particular structures, process steps, or materials disclosed herein, but is extended to equivalents thereof as would be recognized by those ordinarily skilled in the relevant arts. It should also be understood that terminology employed herein is used for the purpose of describing particular embodiments only and is not intended to be limiting.

It must be noted that, as used in this specification and the appended claims, the singular forms “a,” “an,” and “the” include plural referents unless the context clearly dictates otherwise. Thus, for example, reference to a composition containing “a steroid” includes reference to a composition having one or more of such steroids, and reference to “an antibiotic” includes reference to one or more of such antibiotics.

### DEFINITIONS

In describing and claiming the present invention, the following terminology will be used in accordance with the definitions set out below. As used herein, “subject” and “bovine” can be used interchangeably, and refer to a medium-sized to large ungulate, including domestic cattle, Bison, Water Buffalo, the Yak, and the four-horned and spiral-horned antelopes. General characteristics of bovines include a cloven hoof and usually at least one of the sexes of a species having a true horn. As used herein, “composition” refers to a mixture of two or more compounds, elements, or molecules. In some aspects the term “composition” may be used to refer to a mixture of a compound and a pharmaceutically acceptable carrier.

As used herein, the terms “administration,” “administering,” and “delivering” refer to the manner in which a composition is presented to a subject. Administration can be accomplished by various art-known routes such as oral, parenteral, transdermal, inhalation, and implantation. Thus, an oral administration can be achieved by swallowing, chewing, sucking of an oral dosage form comprising the composition. Parenteral administration can be achieved by injecting a composition intravenously, intra-arterially, intramuscularly, intraarticularly, intrathecally, intraperitoneally, subcutaneously, intratumorally, and intracranially. Injectables for such use can be prepared in conventional forms, either as a liquid solution or suspension, or in a solid form that is suitable for preparation as a solution or suspension in a liquid prior to injection, or as and emulsion. Additionally, transdermal administration can be accomplished by applying, pasting, rolling, attaching, pouring, pressing, and rubbing of a transdermal composition onto a skin or hide surface. These and additional methods of administration are well-known in the art. Suitable excipients that can be used for administration include, for example, water, saline, dextrose, glycerol, ethanol, and the like; and if desired, minor amounts of auxiliary substances such as wetting or emulsifying agents, buffers, and the like.

As used herein, the term “co-administer” refers to both the administration of each agent in a sequential manner and the administration of each agent in a simultaneous or substantially simultaneous manner.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

### THE INVENTION

Aspects of the present invention are directed towards methods for treating a variety of mycoplasma-related conditions in bovine animals such as cattle. The inventors have discovered a combination therapy of various compounds that can be used to treat such conditions. In many cases, the physiological symptoms associated with mycoplasma conditions can be reversed, even for those advanced cases previously thought to be generally untreatable. It should be noted that the combination therapy within the present scope can vary somewhat depending on the particular condition being treated, the type of bovine, etc. In one aspect, for example, a combination therapy for the treatment of mycoplasma-related conditions may include administering to a bovine a therapeutically effective amount of a steroid or a pharmaceutically acceptable salt thereof, and a therapeutically effective amount of an antibiotic.

Numerous steroids are contemplated for use in treatments according to aspects of the present invention, and all steroids having a therapeutic effect in the treatment of mycoplasma-related conditions should be considered to be within the present scope. For example, many useful steroids can be categorized as sex steroids, anabolic steroids, and corticosteroids. Non-limiting categorizations of sex steroids include androgens, estrogens, and progestagens. Anabolic steroids such as testosterone are a class of steroids that target androgen receptors to increase muscle and bone synthesis. Additionally, corticosteroids include glucocorticoids and mineralocorticoids.

Glucocorticoids are particularly important in the combination therapies according to aspects of the present invention because they target glucocorticoid receptors and thus act to regulate many aspects of metabolism and immune function. Non-limiting examples of glucocorticoids can include hydrocortisone (cortisol), cortisone acetate, prednisone, prednisolone, methylprednisolone, dexamethasone, betamethasone, triamcinolone, beclometasone, fludrocortisone acetate, deoxycorticosterone acetate, aldosterone, and the like. In one specific aspect, the glucocorticoid steroid is dexamethasone. Dexamethasone is a potent synthetic glucocorticoid. It acts as an anti-inflammatory and immunosuppressant, and has a potency that is about 20-30 times that of hydrocortisone and 4-5 times that of prednisone. It should be noted that one or more glucocorticoids can be used in combination in order to affect treatment of the mycoplasma-related condition. For example, dexamethasone may be administered in combination with another glucocorticoid such as triamcinolone or prednisone.

The route of administration of a steroid can vary depending on the form of the steroid and the condition being treated. The steroid may be administered as an oral, a parenteral, a transdermal, an inhalation, or an implantation formulation. In one specific aspect, the steroid can be delivered as a parenteral formulation in the form of, for example, an intravenous injection, an intramuscular injection, and the like. Additionally, administered dosages of steroids may vary depending on the steroid, the type of animal, and the condition being treated. Such dosing would be readily understood by one of ordinary skill in the art once in possession of the present disclosure. In one specific aspect, however, dexamethasone can be parenterally administered by intravenous injection to an animal in an amount of from about 0.5 mg to about 10.0 mg per 100 lbs of body weight of the animal. In another specific aspect, dexamethasone can be parenterally administered by intravenous injection to an animal in an amount of from about 1.0 mg to about 5.0 mg per 100 lbs of body weight of the animal. In yet another specific aspect, dexamethasone can be parenterally administered by intravenous injection to an animal in an amount of from about 1.5 mg to about 2.5 mg per 100 lbs of body weight of the animal.

Various antibiotics can be utilized in combination with a steroid for the treatment of a mycoplasma-related condition in an animal. Non-limiting examples of broad antibiotic categories can include β-lactams, quinolones, tetracyclines, sulfonamides, fenicoles, macrolides, and the like. The steroid can be administered with a sole antibiotic, or it can be administered with a combination of one or more antibiotics.

A variety of antibiotics are contemplated for use in treatment of mycoplasma-related conditions according to aspects of the present invention. β-lactams are one class of antibiotics that may be useful for such treatment. β-lactams include numerous useful penicillin antibiotics. Non-limiting examples of penicillin antibiotics can include penicillin, benzathine penicillin, benzylpenicillin (penicillin G), phenoxymethylpenicillin (penicillin V), procaine penicillin, oxacillin, meticillin, dicloxacillin, flucloxacillin, temocillin, amoxicillin, ampicillin, co-amoxiclav (amoxicillin+clavulanic acid), azlocillin, carbenicillin, ticarcillin, mezlocillin, piperacillin, nafcillin, cloxacillin, and the like. In one specific aspect, penicillin can be utilized as part of the combination therapy to treat a mycoplasma related condition.

Another useful group of β-lactam antibiotic includes the cephalosporins. Non-limiting examples of cephalosporins can include cefacetrile, cefadroxil, cefalexin, cefaloglycin, cefalonium, cefaloridine, cefalotin, cefapirin, cefatrizine, cefazaflur, cefazedone, cefazolin, cefradine, cefroxadine, ceftezole, cefaclor, cefonicid, cefprozil, cefuroxime, cefuzonam, cefmetazole, cefotetan, cefoxitin, cefamandole, loracarbef, cefbuperazone, cefmetazole, cefminox, cefotetan, cefoxitin, cefcapene, cefdaloxime, cefdinir, cefditoren, cefetamet, cefixime, cefmenoxime, cefodizime, cefotaxime, cefpimizole, cefpodoxime, cefteram, ceftibuten, ceftiofur, ceftiolene, ceftizoxime, ceftriaxone, cefoperazone, ceftazidime, latamoxef, ceftazidime, cefclidine, cefepime, cefluprenam, cefoselis, cefozopran, cefpirome, cefquinome, flomoxef, cefaclomezine, cefaloram, cefaparole, cefcanel, cefedrolor, cefempidone, cefetrizole, cefivitril, cefmatilen, cefmepidium, cefovecin, cefoxazole, cefrotil, cefsumide, ceftaroline, ceftioxide, ceftobiprole, cefuracetime, and the like. In a more specific aspect, useful cephalosporins can include cefadroxil, cefazolin, cefalotin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftobiprole, and the like.

Furthermore, β-lactams can include carbapenems, such as imipenem, meropenem, ertapenem, faropenem, doripenem, and monobactams such as aztreonam. Additionally, β-lactams can be coadministered with a β-lactamase inhibitor, such as clavulanic acid, tazobactam, or sulbactam. Although these β-lactamase inhibitors exhibit negligible antimicrobial activity, they contain a β-lactam ring, and as such may prevent the inactivation of β-lactam antibiotics by binding to β-lactamases.

Other useful antibiotics are additionally contemplated. In one aspect, for example, the antibiotic may include a quinolone such as ciprofloxacin, enoxacin, gatifloxacin, levofloxacin, lomefloxacin, moxifloxacin, norfloxacin, ofloxacin, trovafloxacin, and the like. In another aspect, the antibiotic may include a tetracycline such as demeclocycline, doxycycline, minocycline, oxytetracycline, tetracycline, and the like. In yet another aspect, the antibiotic may include a sulfonamide such as mafenide, prontosil, sulfacetamide, sulfamethizole, sulfanilimide, sulfasalazine, sulfisoxazole, trimethoprim, trimethoprimsulfamethoxazole, and the like. In a further aspect, the antibiotic may include a macrolide such as azithromycin, clarithromycin, dirithromycin, erythromycin, roxithromycin, troleandomycin, telithromycin, spectinomycin, and the like. As additional examples, the antibiotic may also include arsphenamine, chloramphenicol, thiamphenol, florfenicol, clindamycin, lincomycin, ethambutol, fosfomycin, fusidic acid, furazolidone, isoniazid, linezolid, metronidazole, mupirocin, nitrofurantoin, platensimycin, pyrazinamide, quinupristin/dalfopristin, rifampin, tinidazole, tulathromycin, fluoroquinolone, tilmicosin, and the like. As one specific example, tulathromycin can be coadministered with a steroid to the bovine either with or without additional antibiotics. Tulathromycin is a semi-synthetic macrolide antibiotic of the subclass triamilide that is marketed by Pfizer® under the tradename of Draxxin®.

The route of administration of an antibiotic can vary depending on the form of the antibiotic and the condition being treated. The antibiotic may be administered as an oral, a parenteral, a transdermal, an inhalation, or an implantation formulation. In one specific aspect, the antibiotic can be delivered as a parenteral formulation in the form of, for example, an intravenous injection, an intramuscular injection, and the like. Additionally, administered dosages of antibiotics may vary depending on the antibiotic, the type of animal, and the condition being treated. Such dosing would be readily understood by one of ordinary skill in the art once in possession of the present disclosure. For example, in one specific aspect penicillin can be administered by intramuscular injection to an animal in an amount of from about 500,000 units to about 10,000,000 units per 100 lbs of body weight of the animal. In another specific aspect, penicillin can be administered by intramuscular injection to an animal in an amount of from about 1,000,000 units to about 6,000,000 units per 100 lbs of body weight of the animal. In yet another specific aspect, penicillin can be administered by intramuscular injection to an animal in an amount of from about 1,500,000 units to about 4,000,000 units per 100 lbs of body weight of the animal. As another example, florfenicol can be administered by intramuscular injection to an animal in an amount of from about 300 mg to about 3000 mg per 100 lbs of body weight of the animal. In another specific aspect, florfenicol can be administered by intramuscular injection to an animal in an amount of from about 500 mg to about 2200 mg per 100 lbs of body weight of the animal. In yet another specific aspect, florfenicol can be administered by intramuscular injection to an animal in an amount of from about 750 mg to about 1400 mg per 100 lbs of body weight of the animal. As yet another example, sulfadimethoxine can be administered orally to an animal in an amount of from about 10 mg to about 100 mg per 100 lbs of body weight of the animal. In another specific aspect, sulfadimethoxine can be administered orally to an animal in an amount of from about 25 mg to about 75 mg per 100 lbs of body weight of the animal. In yet another specific aspect, sulfadimethoxine can be administered orally to an animal in an amount of from about 40 mg to about 60 mg per 100 lbs of body weight of the animal.

In one aspect, tulathromycin can be administered by parenteral injection, at a dosage of from about 50.0 mg to about 150 mg per 100 lbs of body weight of the animal. In another aspect, tulathromycin can be administered by parenteral injection at a dosage of from about 90.0 mg to about 120 mg per 100 lbs of body weight of the animal.

A variety of combination therapies are additionally contemplated for the treatment of mycoplasma related conditions. In one specific aspect, for example, treatment may include the administration of a steroid and combination of a β-lactam antibiotic and a sulfonamide antibiotic. In a more specific aspect, the β-lactam can be a penicillin antibiotic and the sulfonamide can be sulfadimethoxine. In another specific aspect, chloramphenicol or a chloramphenicol derivative can be administered to treat the condition. The chloramphenicol can be delivered as a sole antibiotic in combination with the steroid, or it can be delivered in combination with the steroid and one or more additional antibiotics, such as a β-lactam, a sulfonamide, and the like. It should be noted that a chloramphenicol derivative is defined as a compound that is recognized by one of ordinary skill in the art as being derived from chloramphenicol. Non-limiting examples include thiamphenicol, which is a methyl-sulfonyl analogue of chloramphenicol, and florfenicol, a fluorenated synthetic of thiamphenicol. In one aspect, tulathromycin can be coadministered as a replacement for, or a supplement to, the chloramphenicol component of the therapy.

It has also been discovered that the combination therapies according to aspects of the present invention may be enhanced by co-administering a vitamin. In one aspect, for example, a vitamin may be administered along with the steroid and one or more antibiotics. Although any vitamin that would be beneficial in the treatment of mycloplasma related conditions would be considered to be within the present scope, non-limiting examples of vitamins can include Vitamin A, Vitamin B1, Vitamin B2, Vitamin B3, Vitamin B5, Vitamin B6, Vitamin B7, Vitamin B9, Vitamin B12, Vitamin C, Vitamin D, Vitamin E, Vitamin K, and complexes and combinations thereof. In one specific aspect, for example, a therapeutically effective amount of a Vitamin B compound can be administered as part of a combination therapy. More specifically, a therapeutically effective amount of a Vitamin B complex can be administered.

In another more specific aspect of the present invention, a method for treating or preventing a mycoplasma-related bovine disease may include administering to a bovine a therapeutically effective amount of dexamethasone or a pharmaceutically acceptable salt thereof, a member selected from the group consisting of florfenicol, thiamphenicol, chloramphenicol, and combinations thereof, a penicillin antibiotic, and sulfadimethoxine. Such a combination therapy can additionally include a vitamin such as a Vitamin B compound or a Vitamin B complex.

The present invention also includes kits containing the various components of a combination therapy, including, in many cases, instructions on dosing and/or administration. In one aspect, for example, a kit may include a steroid, one or more antibiotics, and a set of instructions. As one specific example, the kit may include dexamethasone, one or more antibiotics, such as penicillin, and a set of instructions describing the use of the dexamethasone and the antibiotics. In another specific example, the kit may include dexamethasone, penicillin, at least one additional antibiotic, and a set of instructions. In yet another specific example, the kit may include dexamethasone, penicillin, florfenicol, at least one additional antibiotic, and a set of instructions. In a further specific aspect, the kit may include dexamethasone, penicillin, florfenicol, sulfadimethoxine, and a set of instructions. In various aspects the kit may additionally include a vitamin such as a Vitamin B compound or Vitamin B complex. The kit may be packaged in a housing to contain the components.

### EXAMPLES

The following examples are provided to promote a more clear understanding of certain embodiments of the present invention, and are in no way meant as a limitation thereon.

### Example 1

### Veterinary Inspection of Calves Showing Signs of Mycoplasma-Related Conditions

Four calves showing symptoms of a mycoplasma-related condition were inspected at time=0 by a veterinarian and described as follows:

Calf 1: Calf is down but sternal and had to be assisted to stand. Calf is depressed, and has an occasional cough. Temperature is 103.5° F., heart rate is 96 bpm, and respiration rate is 60 rpm. The left ear droops lower than the right and is rarely voluntarily moved. The calf appears mildly ataxic.

Calf 2: Calf is down but sterna, very depressed. Stands when assisted.

Temp is 105.7° F., heart rate is 100 bpm, and respiration rate is 80 rpm. No nasal discharge, but has some dried discharge on the external ear canal. The calf is ataxic when standing.

Calf 3: Calf is down but sterna, depressed. Stands with assistance. Temperature is 102.8° F., heart rate is 120 bpm, and respiration rate is 52 rpm. Left ear is drooped and the external ear canal has dried exudate. Calf does not move ear, and has very labored respiration.

Calf 4: Calf is standing but depressed, and shakes head frequently. Temperature is 105.6° F., heart rate is 88 bmp, and respiration rate is 120 rpm. The calf does not appear to have a cough or nasal discharge, but has increased respiratory effort.

Calves presented similarly prior to treatment, and the condition was characterized by depression, anorexia, ataxia, pyrexia, cough, head shaking, drooped ears, and ear exudate, generally unilateral. Such calves would generally have a guarded prognosis for survival.

### Example 2

### Treatment of Calves Showing Signs of Mycoplasma-Related Conditions

Beginning at time=0, calves 1-4 from Example 1 were treated as follows, with the dosages being per 100 lb of body weight:

5 cc intramuscular injection of 300,000 unit penicillin G potassium;

3.5 cc intramuscular injection of Nuflor®;

Intravenous delivery of 5 cc of 100 mg vitamin B complex and 1 cc of 2 mg dexamethasone;

50 mg of Albon® bolus delivered orally.

### Example 3

### Veterinary Inspection of Treated Calves Showing Signs of Mycoplasma-Related Conditions

Calves from Example 1 were again inspected following the treatment according to Example 2 by the veterinarian at approximately time=2.5 hours as follows:

Calf 1: The calf is standing and more alert. No cough is noted. Temperature is 102.8° F., heart rate is 120 bpm, and respiration rate is 52 rpm. No ataxia noted. The left ear still droops lower than the right and is rarely voluntarily moved.

Calf 2: The calf is standing and drinking water, alert. No cough noted. Temperature is 104.8° F., heart rate is 160 bpm, and respiration rate is 60 rpm. Right ear still droops, but the calf is much brighter. No ataxia noted.

Calf 3: The calf is standing and resisting exam. Has normal respiratory effort. Temperature is 102.0° F., heart rate is 124 bpm, and respiration rate is 36 rpm. Both ears are carried high and are moved frequently.

Calf 4: The calf is standing, and is brighter. The calf's head is shaking now only occasionally. Occasional cough. Temperature is 104.7° F., heart rate is 100 bpm, and respiration rate is 80 rpm.

The calves showed decreased temperatures and became stronger and more alert between the Example 1 inspection and the Example 3 inspection, following the treatment as in Example 2. Respiratory rates slowed, coughs decreased in frequency, and ear carriage improved significantly. Hear rates increased somewhat.

### Example 4

### Supplemental Treatment of Calves Showing Signs of Mycoplasma-Related Conditions

The calves from Example 3 were treated at time=24 hrs as follows, with the dosages being per 100 lb of body weight

5 cc intramuscular injection of 300,000 unit penicillin G potassium;

Intravenous delivery of 5 cc of 100 mg vitamin B complex;

50 mg of Albon® bolus delivered orally.

### Example 5

### Veterinary Inspection of Treated Calves Showing Signs of Mycoplasma-Related Conditions

Calves from Example 1 were again inspected following the treatment according to Example 4 by the veterinarian at approximately time=27 hours as follows:

Calf 1: The calf is standing, is alert, and is eating. No cough, head tilt or drooped ear is noted. Heart rate is 80 bpm and respiration rate is 80 rpm. Respiration is normal, and the calf is very bright.

Calf 2: The calf is standing, is alert, and is hungry. No abnormalities noted. Temperature is 101.1° F., heart rate is 120 bpm, and respiration rate is 48 rpm. No cough or ear droop is noted.

Calf 3: The calf is standing and alert, and no abnormalities are noted. The calf is very vigorous. Temperature is 101.3° F., heart rate is 124 bpm, and respiration rate is 48 rpm.

Calf 4: The calf is bright, alert, and very vigorous. Has a slight head shake, but has no cough and no ear droop. Heart rate is 112 bpm, and respiration rate is 48 rpm.

27 hours following the treatment of Example 2, calves 1-4 showed remarkable improvement. All were bright, alert, vigorous, and exploring their environments. All were also acting very strongly. The only residual problem that was observed was a slight and very occasional head shake in calf 4.

It is to be understood that the above-described embodiments are only illustrative of the applications of the principles of the present invention. Numerous modifications and alternative embodiments can be derived without departing from the spirit and scope of the present invention and the appended claims are intended to cover such modifications and arrangements. Thus, while the present invention has been fully described above with particularity and detail in connection with what is presently deemed to be the most practical and preferred embodiment(s) of the invention, it will be apparent to those of ordinary skill in the art that numerous modifications can be made without departing from the principles and concepts of the invention as set forth in the claims.

We claim:

1. A method for treating or preventing a mycoplasma-related bovine disease, comprising:
administering to a bovine a therapeutically effective amount of a steroid or a pharmaceutically acceptable salt thereof; and
administering to the bovine a therapeutically effective amount of an antibiotic.

2. The method of claim 1, wherein the steroid is a glucocorticoid steroid.

3. The method of claim 2, wherein the glucocorticoid steroid includes a member selected from the group consisting of hydrocortisone, cortisone acetate, prednisone, prednisolone, methylprednisolone, dexamethasone, betamethasone, triamcinolone, beclometasone, fludrocortisone acetate, deoxycorticosterone acetate, aldosterone, and combinations thereof.

4. The method of claim 2, wherein the glucocorticoid steroid is dexamethasone.

5. The method of claim 1, wherein the antibiotic includes a member selected from the group consisting of β-lactams, quinolones, tetracyclines, sulfonamides, fenicoles, macrolides, and combinations thereof.

6. The method of claim 5, wherein the antibiotic is a penicillin antibiotic.

7. The method of claim 1, wherein administering a therapeutically effective amount of an antibiotic further includes administering a β-lactam antibiotic and a sulfonamide antibiotic.

8. The method of claim 7, wherein the β-lactam is a penicillin antibiotic and the sulfonamide is sulfadimethoxine.

9. The method of claim 1, further comprising administering to the bovine a therapeutically effective amount of a member selected from the group consisting of florfenicol, thiamphenicol, chloramphenicol, and combinations thereof.

10. The method of claim 1, further comprising administering to the bovine a therapeutically effective amount of tulathromycin.

11. The method of claim 1, further comprising administering to the bovine a therapeutically effective amount of a Vitamin B compound.

12. A method for treating or preventing a mycoplasma-related bovine disease, comprising:
administering to a bovine a therapeutically effective amount of:
dexamethasone or a pharmaceutically acceptable salt thereof;
a member selected from the group consisting of florfenicol, thiamphenicol, chloramphenicol, tulathromycin, and combinations thereof;
a penicillin antibiotic; and
sulfadimethoxine.

13. The method of claim 12, wherein tulathromycin is administered to the bovine.

14. The method of claim 11, further comprising administering to the bovine a therapeutically effective amount of a Vitamin B compound.

15. A kit for treating or preventing a mycoplasma-related bovine disease, comprising:
a housing containing:
a steroid;
at least one antibiotic; and
a set of instructions describing a use of the steroid and the at least one antibiotic to treat or prevent the mycoplasma-related bovine disease.

16. The kit of claim 15, wherein the steroid is dexamethasone.

17. The kit of claim 16, wherein the at least one antibiotic includes a β-lactam.

18. The kit of claim 16, wherein the at least one antibiotic includes a member selected from the group consisting of florfenicol, tulathromycin, and combinations thereof.

19. The kit of claim 16, wherein the at least one antibiotic includes sulfadimethoxine.

20. The kit of claim 15, further comprising a Vitamin B complex.
